(19) United States
(12) Patent Application Publication
(10) Pub. No.: US 2010/0113915 A1
(43) Pub. Date: May 6, 2010

(54) ORTHOGONALLY POSITIONED TAGGING IMAGING METHOD FOR ARTERIAL LABELING WITH FAIR

(76) Inventors: Richard W. Briggs, Dallas, TX (US); Xiufeng Li, Dallas, TX (US); Kaundinya S. Gopinath, Dallas, TX (US); Subhendra N. Sarkar, Dallas, TX (US)

Correspondence Address: FULBRIGHT & JAWORSKI L.L.P., 600 CONGRESS AVE., SUITE 2400, AUSTIN, TX, 78701, US

(21) Appl. No.: 12/610,845

(22) Filed: Nov. 2, 2009

Related U.S. Application Data
(60) Provisional application No. 61/110,457, filed on Oct. 31, 2008.

(51) Int. Cl. A61B 5/055

(52) U.S. Cl. 600/419

(57) ABSTRACT

Methods, devices, and systems for creating an MRI image using an orthogonally positioned tagging imaging method for arterial labeling with FAIR. Embodiments of the present methods for creating an MRI image may include positioning a perfusion imaging plane that corresponds to an image target area of an imaging object, and causing an MRI image to be generated that corresponds to a representation of the image target area at the perfusion imaging plane. The perfusion imaging plane may be orthogonal to a direction of inflow from immediately proximal arteries of the image target area.

### CROSS-REFERENCE TO RELATED APPLICATIONS

This application claims the benefit of U.S. Provisional Application No. 61/110,457 filed Oct. 31, 2008, the entire contents of which is specifically incorporated herein by reference without disclaimer. This application is also related to commonly owned and co-filed U.S. Application No. ______ filed Nov. 2, 2009, which claims the benefit of U.S. Provisional Application No. 61/110,548, filed Oct. 31, 2008, the entire contents of each of which is specifically incorporated herein by reference without disclaimer.

### STATEMENT REGARDING FEDERALLY SPONSORED RESEARCH OR DEVELOPMENT

This invention was made with government support under Contract No. DAMD17-01-1-0741 awarded by DoD/U.S. Army Med. Res. Acq'n. Activity. The government has certain rights in the invention.

### BACKGROUND OF THE INVENTION

The present methods, devices, and systems relate generally to medical imaging. More particularly, the present methods, devices, and systems relate to orthogonally positioned tagging imaging methods for arterial labeling with FAIR.

### SUMMARY OF THE INVENTION

Embodiments of the present methods for creating an MRI image may include positioning a perfusion imaging plane that corresponds to an image target area of an imaging object, and causing an MRI image to be generated that corresponds to a representation of the image target area at the perfusion imaging plane. The perfusion imaging plane may be orthogonal to a direction of inflow from immediately proximal arteries of the image target area.

Some embodiments may further include receiving data corresponding to the direction of inflow. Some embodiments may further include determining the direction of inflow. Some embodiments may further include receiving data corresponding to the perfusion imaging plane. Some embodiments may further include determining the perfusion imaging plane.

In some embodiments, the imaging target area may include cerebrovascular anatomy. In some embodiments, the cerebrovascular anatomy may include a portion of the cerebellum. In some embodiments, the cerebrovascular anatomy may include a portion of the hippocampus.

Embodiments of the present systems may include an imaging unit, and a controller unit The controller unit may be configured to be operative to position a perfusion imaging plane that corresponds to an image target area of an imaging object, and to cause an MRI image to be generated that corresponds to a representation of the image target area at the perfusion imaging plane. The perfusion imaging plane may be orthogonal to a direction of inflow from immediately proximal arteries of the image target area.

Some embodiments may be configured to be configured to be further operative to receive data corresponding to the direction of inflow. Some embodiments may be configured to be further operative to determine the direction of inflow. Some embodiments may be configured to be further operative to receive data corresponding to the perfusion imaging plane. Some embodiments may be configured to be further operative to determine the perfusion imaging plane.

In some embodiments, the imaging target area may include cerebrovascular anatomy. In some embodiments, the cerebrovascular anatomy may include a portion of the cerebellum. In some embodiments, the cerebrovascular anatomy may include a portion of the hippocampus.

Embodiments of the present computer readable medium may have computer usable program code executable to perform operations that include determining a perfusion imaging plane that corresponds to an image target area of an imaging object, and sending an output that is configured to cause an MRI image to be generated that corresponds to a representation of the image target area at the perfusion imaging plane. The perfusion imaging plane may be orthogonal to a direction of inflow from immediately proximal arteries of the image target area.

In some embodiments, the operations may further include receiving data corresponding to the direction of inflow. In some embodiments, the operations may further include determining the direction of inflow. In some embodiments, the operations may further include receiving data corresponding to the perfusion imaging plane. In some embodiments, the operations may further include determining the perfusion imaging plane.

In some embodiments, the imaging target area may include cerebrovascular anatomy. In some embodiments, the cerebrovascular anatomy may include a portion of the cerebellum. In some embodiments, the cerebrovascular anatomy may include a portion of the hippocampus.

The term “coupled” is defined as connected, although not necessarily directly, and not necessarily mechanically.

The terms “a” and “an” are defined as one or more unless this disclosure explicitly requires otherwise.

The term “substantially” and its variations are defined as being largely but not necessarily wholly what is specified as understood by one of ordinary skill in the art, and in one non-limiting embodiment “substantially” refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Other features and associated advantages will become apparent with reference to the following detailed description of specific embodiments in connection with the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: S-I and A-P transit time differences in the cerebellum.

FIG. 2: Spatial definitions of OPTIMAL FAIR pulse sequence components.

FIG. 3: Perfusion weighted images (top left), CBF maps (top right), and co-registered anatomy (bottom left) from a typical subject from quantitative cerebellum perfusion studies using OPTIMAL FAIR.

FIG. 4: CBF maps using traditional FAIR.

FIG. 5: Slice position used for hippocampus study.

FIG. 6: Perfusion-weighted MDS OPTIMAL FAIR imaging maps of hippocampus.

FIG. 7: System for creating an MRI image using an orthogonally positioned tagging imaging method for arterial labeling with FAIR.

FIG. 8: A computer system adapted according to certain embodiments of the controller unit.

FIG. 9: The CPU 202 may execute machine-level instructions according to the exemplary operations.

### DETAILED DESCRIPTION

The invention and the various features and advantageous details are explained more fully with reference to the non-limiting embodiments that are illustrated in the accompanying drawings and detailed in the following description. Descriptions of well known starting materials, processing techniques, components, and equipment are omitted so as not to unnecessarily obscure the invention in detail. It should be understood, however, that the detailed description and the specific examples, while indicating embodiments of the invention, are given by way of illustration only and not by way of limitation. Various substitutions, modifications, additions, and/or rearrangements within the spirit and/or scope of the underlying inventive concept will become apparent to those skilled in the art from this disclosure.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Transit time and exchange time are critical contributing factors for accurate quantitative perfusion measurements using magnetic resonance imaging (MRI) arterial spin labeling (ASL) techniques. The more uniform transit time and exchange time are within the acquired perfusion imaging slice, the better the quantitative estimation of regional blood flow. The specifics of the cerebrovascular anatomy of some brain regions, such as cerebellum, result in unique blood supply features that dictate specially designed imaging schemes for accurate rCBF quantification and mapping. The general principle is that perfusion imaging slabs/slices should be positioned perpendicular or orthogonal to the direction of inflow from the immediately proximal arteries. For example, to achieve more uniform transit time and exchange time within the imaging slice, a coronal imaging slab/slice may be preferred for cerebellum, since the cerebellar blood supply is mainly via three pairs of feeding arteries (superior cerebellar artery (SCA), anterior inferior cerebellar artery (AICA), and posterior inferior cerebellar artery (PICA) that course primarily anterior to posterior. Since blood in the major carotid and basilar arteries feeding the cerebellar arteries flows inferior to superior, axial slabs may be used for blood labeling. This principle may also be valuable for accurate and more uniform estimates within the imaging slice of other important physiological parameters, such as transit time and exchange time, which can be altered in disease states. By using a relatively larger field of view, the superior labeling band of traditional FAIR can be placed outside the brain, thus removing the possible undesired superior labeling effects of traditional FAIR. Furthermore, partial volume effects can be minimized by using high in-plane resolution of the oblique coronal (or other non-axial) slices, making ROI-based analysis much easier. This technique may be useful in, for example, cerebellum, occipital lobe, and hippocampus perfusion studies, among others.

### Optimal Fair Technique Applied to Cerebellum

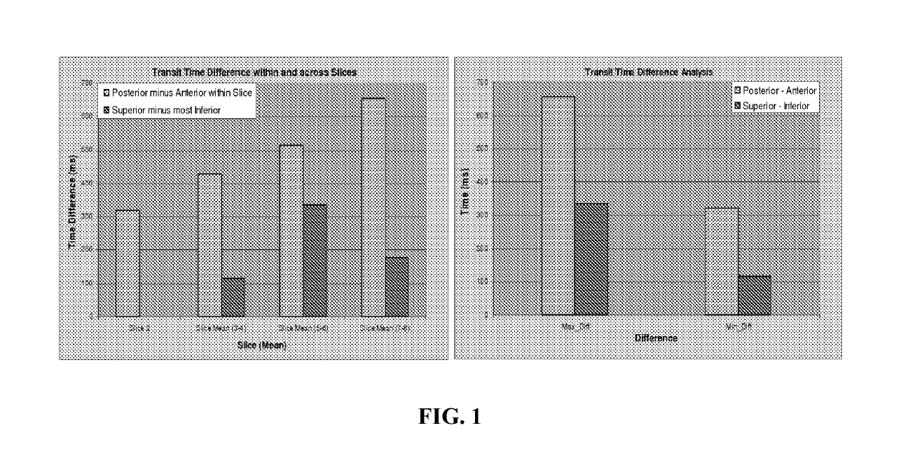

Multiple inversion experiments with axial image slices indicated that the transit time difference between the superior and inferior cerebellum is consistently smaller than that between the anterior and posterior cerebellum (FIG. 1). This suggested that coronal slices through the cerebellum obtained with the present OPTIMAL FAIR embodiments would provide more accurate quantitative perfusion estimations than possible with traditional axial slices, since better uniformity of transit time can thus be obtained with coronal image slices than with conventional axial image slices.

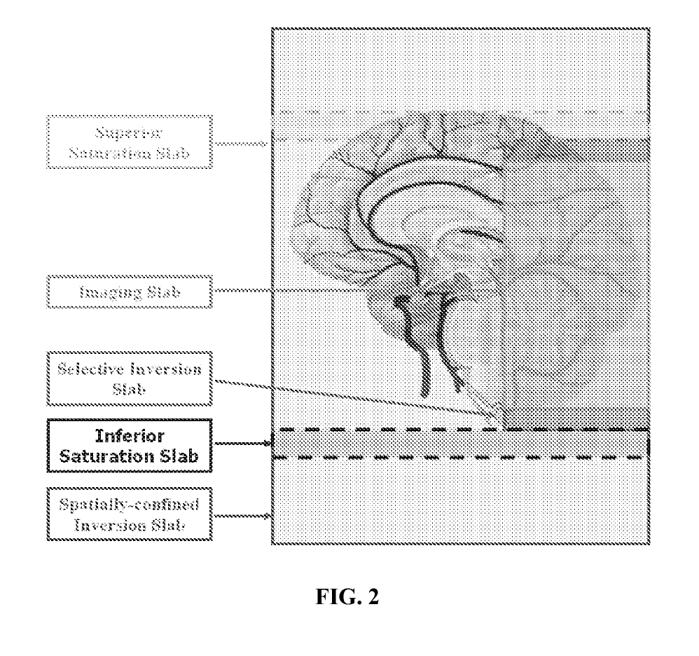

Present OPTIMAL FAIR embodiments were implemented with passive suppression of venous artifacts using modulated dual saturation (MDS), for perfusion studies of the cerebellum. MDS refers to two interleaved periodic saturation pulses performed at the superior side and inferior side of imaging slab. The inferior saturation pulse may better define a temporal bolus width for labeled blood from the inferior side, while the superior saturation pulse train may suppress the labeled venous blood and associated artifacts. For quantification of rCBF, Q2TIPs was also incorporated into this embodiment. For quantification of rCBF, Q2TIPs was also incorporated into this embodiment. The spatial definitions of RF pulses for present embodiments MDS OPTIMAL FAIR are shown in FIG. 2.

FIG. 2 shows spatial definitions of OPTIMAL FAIR pulse sequence components. Brain picture is from Haines, D. E., Neuroanaotmy An atlas of Structures, Sections, and Systems, 6th edition., Lippincott Williams & Wilkins A Wolters Kluwer Company, 2004. To remove the transition effects of selective inversion, the selective inversion slab may be a little larger (10 mm on each side) than the field of view.

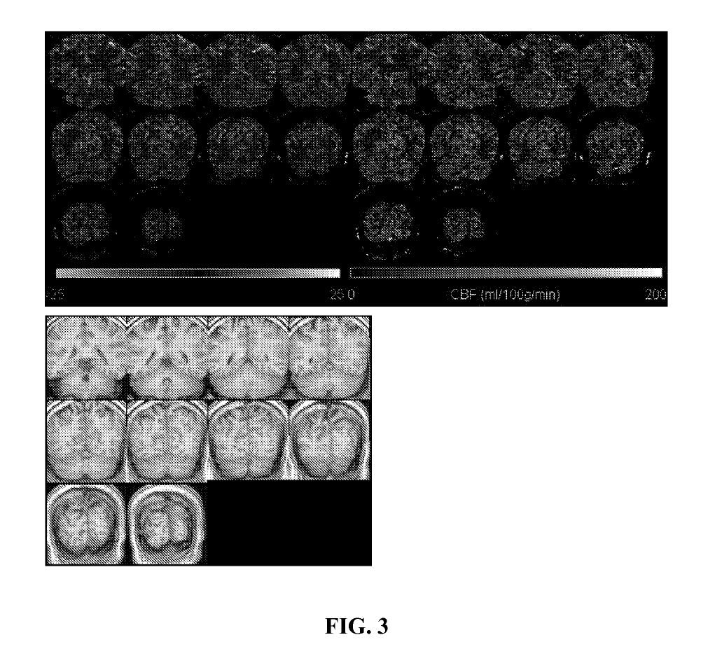

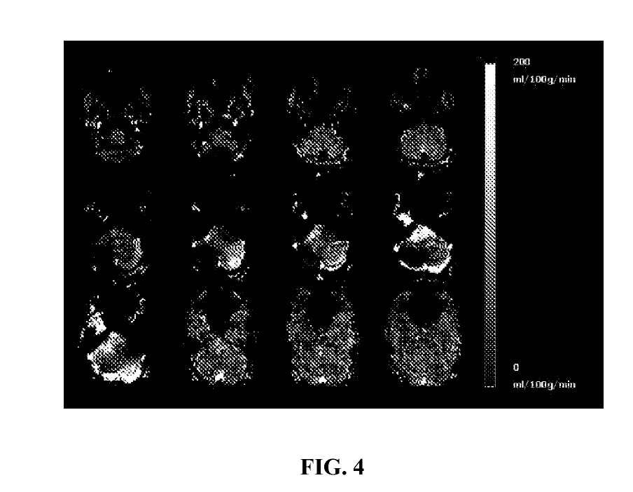

Results of quantitative blood flow studies of cerebellum using these high in-plane resolution (2.5 mm×2.5 mm) OPTIMAL FAIR embodiments are presented in FIG. 3. Venous artifacts are notably absent in the OPTIMAL FAIR images, but dramatically visible as bright regions in the traditional FAIR images shown in FIG. 4.

FIG. 3 shows perfusion weighted images (top left), CBF maps (top right), and co-registered anatomy (bottom left) from a typical subject from quantitative cerebellum perfusion studies using OPTIMAL FAIR. FOV=128×128 mm², matrix size=64×64, number of imaging slices=10, slice thickness/gap=5/1 mm, resolution=2×2×5 (+1 mm gap) mm³; TR/TE=2500/14 ms, 20% phase over sampling, partial Fourier (PF)=6/8, iPAT GRAPPA factor=2 with 24 reference lines, phase encoding direction=left to right, slice acquisition order along anterior to posterior direction, selective labeling size=150 mm, spatially-confined inversion slab size=230 mm, 100 pairs of control and labeling measurements, TI₁/TI₂=800/1000 ms, 20 inferior saturation pulses with 20 mm slab size and 25 ms interval, superior saturation pulse train turned off.)

FIG. 4 shows CBF maps (2.5×2.5×3.5 mm 3) using traditional FAIR with the following MRI parameters: TR/TE=2500/12 ms, field of view (FOV)=180×180 mm², matrix size=72×72, slice thickness/gap=3.5/0.7 mm, the number of imaging slices=16, imaging resolution=2.5×2.5×3.5 (+0.7) mm³, number of measurements=180, iPAT GRAPPA factor=2 with 24 reference lines using CP mode, partial Fourier (PF)=7/8, acquisition order=ascending (foot to head), imaging section inversion slab size=imaging slab size+20 mm, spatially-confined inversion slab size=imaging slab size+200 mm, temporal bolus width (TI₁)/post-bolus delay=800/1000 ms, inferior saturation number=20 with 25 ms interval using 20 mm saturation slab.

Embodiments of OPTIMAL FAIR may optimally place slice orientation orthogonal or perpendicular to the proximal feeding arterial direction for cerebellum, producing more reliable CBF estimates due to increased homogeneity of transit time within slices, and effectively avoiding venous artifacts.

### Optimal Fair Technique Applied to Hippocampus

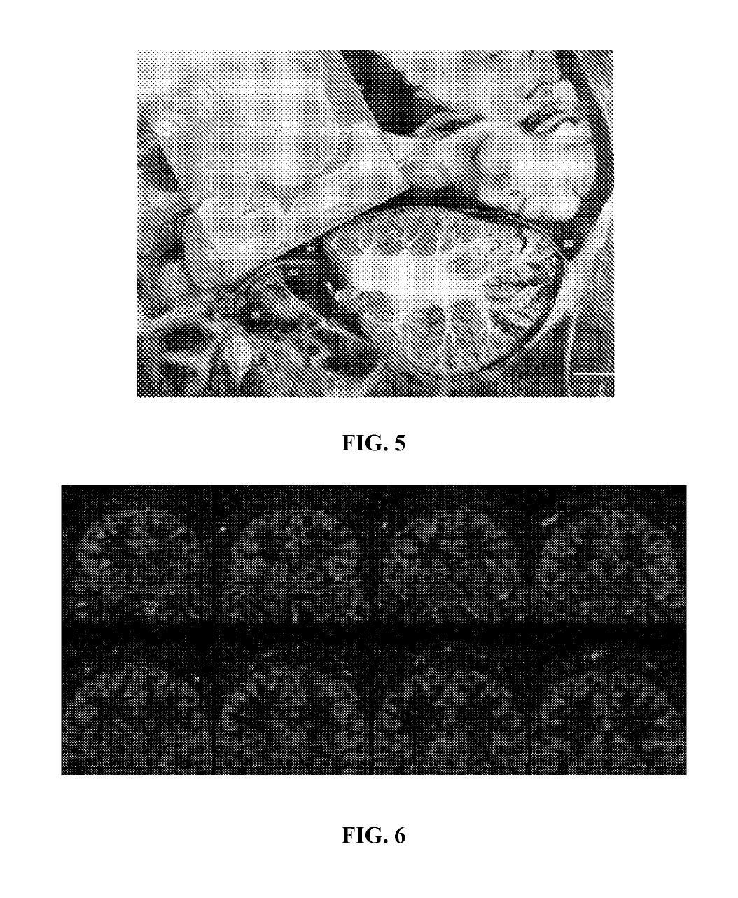

The blood supply for the hippocampus is mainly via branches from the posterior cerebral arteries, and to a lesser degree from the anterior choroidal arteries. For the hippocampus body and tail, blood supplies are completely via medial and lateral posterior choroidal arteries from the posterior cerebral arteries, which are parallel to the long axis of the hippocampus, with arterial flow from anterior to posterior. Thus, based on these cerebrovascular anatomy considerations, the transit time is hypothesized to be longer in the tail than in the body. For the hippocampus head, blood supply is also from branches arising from the anterior choroidal artery, whose source is the internal carotid artery, which may further shorten the transit time for the hippocampus head. Therefore, the acquisition of oblique coronal slices for the hippocampus, from anterior to posterior may be desirable. To follow the major proximal arterial input blood flow direction (at least for hippocampus body and tail), which also minimizes partial volume effects, oblique coronal images collected temporally anterior to posterior were used in a hippocampus perfusion study. The slice positions for this study are shown in FIG. 5.

FIG. 5 shows slice position used for hippocampus study. Figure from Duvernoy, H. M., The Human Hippocampus Functional Anatomy, Vascularization and Serial Sections with MRI. 3^{rd} edn., Springer-Verlag, Berlin, 2005.

Initial quantitative hippocampus perfusion studies using a present OPTIMAL FAIR embodiment have been performed with several post-bolus delays. An example from these studies is shown in FIG. 6.

FIG. 6 shows perfusion-weighted MDS OPTIMAL FAIR imaging maps of hippocampus. Parameters are FOV=220×220 mm², matrix size=110×110, slice thickness/gap=5/1 mm, resolution=2×2×5 (+1) mm³, TR/TE=2500/14 ms, iPAT GRAPPA factor=2 with 24 reference lines and CP mode, PF=6/8, acquisition order=ascending (from anterior side to posterior side), 20% phase over sampling, temporal bolus width/post-bolus delay=600/1200 ms, inferior saturation number=40 with 20 mm slab size and 25 ms interval.

### Prior Technology

Prior software programs (and methods) for pulsed arterial labeling similar to the proposed OPTIMAL FAIR technique for quantitative studies include traditional FAIR techniques, PICORE and TILT with QUIPSS II and QUIPSS II Tips (Luh, W. M., et al., Magn Reson Med. 41(6): 1246-1254 (1999).

One aspect by which the present OPTIMAL FAIR embodiments differs from the prior technology is that the OPTIMAL FAIR embodiments are specially configured and optimized for specific cerebrovascular anatomy. For example, in embodiments of OPTIMAL FAIR the acquisition slab/slice orientation may be orthogonal or perpendicular to the major proximal arteries feeding the region whenever the labeled arteries (typically carotids or basilar) are not parallel to the feeding arteries providing primary inflow to the region of interest. This may permit more accurate and reliable quantitative or semi-quantitative perfusion studies, due to a more uniform transit time distribution throughout the imaged slice. By acquiring imaging slices along the direction of blood flow in these primary proximal feeding arteries, transit times can be made relatively uniform in the different slices in the imaging slab.

### Features of the Present Embodiments

The present OPTIMAL FAIR embodiments may provide increased uniformity in transit time and exchange time within the imaging slice(s) in MR arterial spin labeling applications in specific regions, such as cerebellum and hippocampus, where the proximal feeding arteries are not parallel to the larger upstream arteries (typically carotid or basilar arteries) labeled in the experiment.

Routine ASL as typically implemented with FAIR may have rather low spatial resolution, and exhibit prominent vascular artifacts in inferior and temporal aspects of brain. Embodiments of OPTIMAL FAIR demonstrate that excellent visualization and quantification of CBF can be done in these regions. Within-slice estimates of blood flow may be rendered more uniform, and hemispheric asymmetries may be reduced. Since many radiological interpretations depend on qualitative, fast visualization of perfusion, this embodiments of the present disclosure may help clinicians to more reliably assess ipsilateral versus contralateral perfusion effects of tumor or stroke.

Cerebellum is less studied by ASL than peripheral cortical regions, but is important in brain stem strokes, autism, and gait and movement disorders. Hippocampus is a small, complex organ with a functionally and anatomically complicated vascular system. Embodiments of the present OPTIMAL FAIR techniques may significantly improve the qualitative appearance and quantitative accuracy of perfusion maps for both, for medium- and high-resolution ASL. Embodiments of the present disclosure may offer similar advantages for other brain regions or other parts of the body.

Embodiments of the present disclosure may be suitable for quantitative perfusion studies of regions where the vascular architecture provides non-parallel orientations between the labeled artery and the immediate feeding artery providing inflow to the cerebral territory in the imaged slice. The present embodiments can be combined with other arterial spin labeling techniques, such as TILT, PICORE, CASL, and pCASL. For cerebellum studies using larger fields of view, the present embodiments may also effectively remove superior venous contamination that would normally be seen with traditional axial slice FAIR. In addition, artifacts produced by blood flowing in the A-P surface veins of the cerebellum that generate phase errors that may produce motion-related signal modulations in axial slices may not be produced in the coronal slices provided by embodiments of the present disclosure.

### Problems Addressed by the Present Embodiments

OPTIMAL FAIR embodiments may solve the problem of non-uniform transit time and exchange time within the imaging slice in cases where the vascular anatomy results in orthogonal or non-parallel orientations between the labeled major artery and the primary feeding artery providing inflow to the brain in the imaged slice. This may enable more accurate quantitative perfusion studies to be performed using pulsed arterial spin labeling, as demonstrated for cerebellum. At the same time, using larger coronal or other non-axial field of view, venous blood artifacts from labeling superior to the image slab and flow modulations can also be removed.

Although the embodiments of the present disclosure serve the useful function of improving the CBF quantification in perfusion studies of brain, they can also be useful for perfusion studies in other organs or regions where similar considerations arise when attempting accurate and reliable CBF quantification. Furthermore, some present embodiments may include other ASL techniques, such as PICORE, pseudo-continuous ASL (pCASL) and TILT (to yield OPTIMAL PICORE, OPTIMAL pCASL, OPTIMAL TILT).

The present embodiments may be employed by, for example, MRI physicists and neuroimaging scientists with MRI backgrounds and arterial spin labeling knowledge for “quantitative” perfusion analysis and adapting protocols to specific subject populations (e.g., stroke and dementia) and regions of interest. Potential uses include pre-clinical, translational, and neuropsychiatric applications and research conducted by psychiatrists, neurologists, neuroradiologists, pathologists, neurosurgeons, and medical scientists. Additionally, diagnostic imaging to “qualitatively” detect perfusion deficits in less than half an hour can be performed by MR technologists supervised by neuroradiologists.

All the sequences were tested on phantoms before initial subject scans. The initial subject scans were conducted on May 1, 2007 at the 3T Siemens MR scanner in the Neuroimaging Lab, Room MG.119, Meadows MRI Center, UT Southwestern Medical Center, Dallas Tex. For results, see the attachments.

Embodiments of the present OPTIMAL FAIR technique were initially used for cerebellum perfusion studies in a human subject. Since then, present OPTIMAL FAIR embodiments have been used in 15 additional subjects in cerebellum studies and 12 subjects in hippocampus. In all cases, embodiments of the present software were used on the 3T Siemens MR scanner at the Neuroimaging Lab, Room MG.119, Meadows MRI Center, UT Southwestern Medical Center, Dallas, Tex.

### Overcoming Current Supporting Technology Limitations

The default minimal matrix size in the supporting technology currently employed prevents the present embodiment's sequence from prescribing a smaller field of view to allow better spatial resolution and shorter TE. Since some brain regions have large susceptibility effects that can be minimized by using shorter TE, it may be advantageous to include methods that offer limited fields of view, such as zoomed EPI (Pfeuffer, J., et al., NeuroImage 17: 272-286 (2002)) in some embodiments of the present disclosure, to permit advanced perfusion studies with higher resolution in those areas.

Due to Ti relaxation, the number of slices and thus coverage may be limited.

Due to the interference between the imaging and labeling of present OPTIMAL FAIR embodiments, labeling efficiency can be affected, for example, in the cerebellum perfusion studies of whole cerebellum, but this can be corrected or calibrated.

### Depicted Embodiments

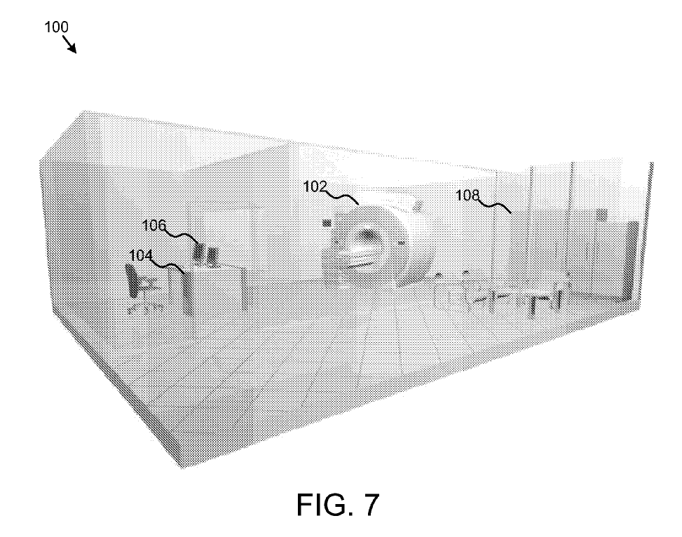

FIG. 7 illustrates one embodiment of a system 100 for creating an MRI image using an orthogonally positioned tagging imaging method for arterial labeling with FAIR. The system 100 may include an image capture device 102 and a controller unit 104. In a further embodiment, the system 100 may include a user interface 106 and other support devices 108, including power sources, data storage devices, networking devices, image processing devices, etc.

In the depicted embodiment, the image capture device 102 may include a magnetic resonance (MR) medical imaging device. For example, the present embodiments may include a 3T Siemens® Trio TIM whole-body scanner. The image capture device may further include a 60 cm diameter magnet bore and SQ gradients (maximum gradient strength 45 mT/m in the z direction and 40 mT/m in the x and y directions, maximum slew rate 200 mT/m.ms, 200 μs rise time). The image capture device may include one or more magnets, one or more gradient magnets or coils, and one or more Radio Frequency (RF) coils. In a particular embodiment, the image capture device 102 may include 12-channel phased array detector coils.

The controller unit 104 may include a software application, program, process, or algorithm configured to generate control signals for the imaging device 102. The controller unit 104 may then communicate the control signals to the imaging device 102. For example, the controller unit 104 may communicate control signals configured to control the polarity or magnetic orientation produced by the gradient coils. The control unit 104 may also control the frequency and timing of the RF coils. In a further embodiment, the control unit 104 may control patient positioning. The control signals may further control phase angles of the 12-channel phased array detector coils. In such embodiments, the controller unit 104 may generate the control signals in response to one or more imaging sequence parameters. The imaging sequence parameters may be user inputs, selectable controls, or a calculated result of user inputs or controls.

In a further embodiment, the control unit 104 may process signals received from the imaging unit 102. For example, the control unit 104 may generate images from data received from the imaging unit 102. In further embodiments, the control unit 104 may filter, enhance, color, or otherwise process the resulting images in response to one or more user selections or predefined processes.

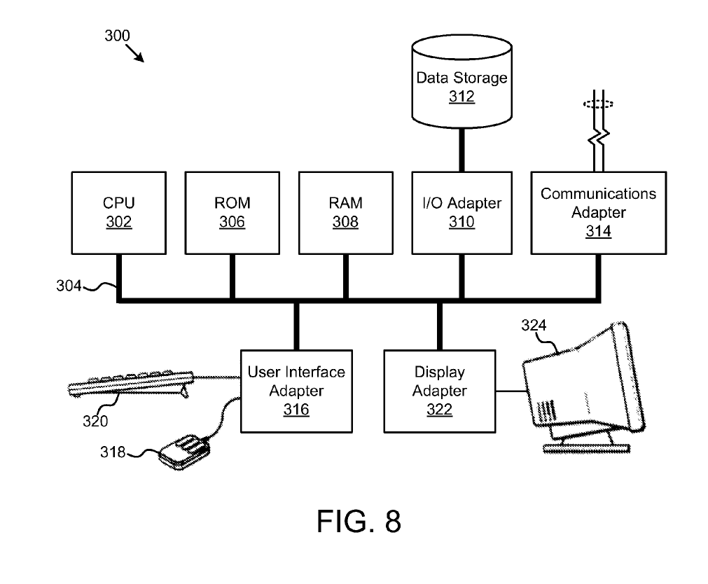

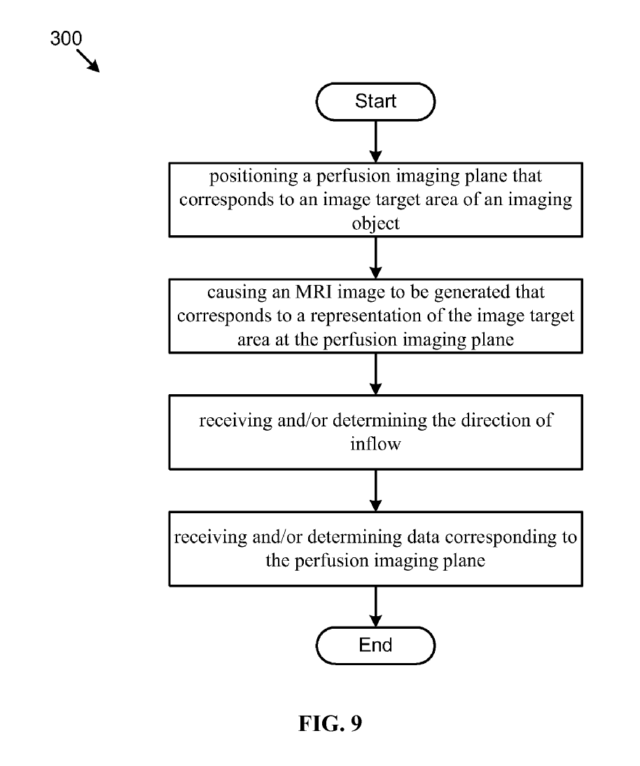

FIG. 8 illustrates a computer system 200 adapted according to certain embodiments of the controller unit 104. The central processing unit (CPU) 202 is coupled to the system bus 204. The CPU 202 may be a general purpose CPU or microprocessor. The present embodiments are not restricted by the architecture of the CPU 202, so long as the CPU 202 supports the modules and operations as described herein. The CPU 202 may execute the various logical instructions according to the present embodiments. For example, the CPU 202 may execute machine-level instructions according to the exemplary operations described below with reference to FIG. 9.

The computer system 200 also may include Random Access Memory (RAM) 208, which may be SRAM, DRAM, SDRAM, or the like. The computer system 200 may utilize RAM 208 to store the various data structures used by a software application configured to create an MRI image using an orthogonally positioned tagging imaging method for arterial labeling with FAIR. The computer system 200 may also include Read Only Memory (ROM) 206 which may be PROM, EPROM, EEPROM, or the like. The ROM may store configuration information for booting the computer system 200. The RAM 208 and the ROM 206 hold user and system 100 data.

The computer system 200 may also include an input/output (I/O) adapter 210, a communications adapter 214, a user interface adapter 216, and a display adapter 222. The I/O adapter 210 and/or user the interface adapter 216 may, in certain embodiments, enable a user to interact with the computer system 200 in order to input information for authenticating a user, identifying an individual, or receiving health profile information. In a further embodiment, the display adapter 222 may display a graphical user interface associated with a software or web-based application for presenting a natural history of a disease.

The I/O adapter 210 may connect to one or more storage devices 212, such as one or more of a hard drive, a Compact Disk (CD) drive, a floppy disk drive, a tape drive, to the computer system 200. The communications adapter 214 may be adapted to couple the computer system 200 to the imaging unit 102. The user interface adapter 216 couples user input devices, such as a keyboard 220 and a pointing device 218, to the computer system 200. The display adapter 222 may be driven by the CPU 202 to control the display on the display device 224.

The present embodiments are not limited to the architecture of system 200. Rather the computer system 200 is provided as an example of one type of computing device that may be adapted to perform the functions of the controller unit 104. Moreover, the present embodiments may be implemented on digital signal processor (DSPs), application specific integrated circuits (ASIC) or very large scale integrated (VLSI) circuits. In fact, persons of ordinary skill in the art may utilize any number of suitable structures capable of executing logical operations according to the described embodiments.

The schematic flow chart diagrams that follow are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

FIG. 8 illustrates one embodiment of a method 300 for creating an MRI image using an orthogonally positioned tagging imaging method for arterial labeling with FAIR.

All of the methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope of the invention. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the invention as defined by the appended claims.

Further disclosure of methods, devices, and systems related to some examples of the present disclosure is provided in Appendix.

What is claimed is:

1. A method for creating an MRI image, the method comprising:
positioning a perfusion imaging plane that corresponds to an image target area of an imaging object, where the perfusion imaging plane is orthogonal to a direction of inflow from immediately proximal arteries of the image target area;
causing an MRI image to be generated that corresponds to a representation of the image target area at the perfusion imaging plane.

2. The method of claim 1, further comprising receiving data corresponding to the direction of inflow.

3. The method of claim 1, further comprising determining the direction of inflow.

4. The method of claim 1, further comprising receiving data corresponding to the perfusion imaging plane.

5. The method of claim 1, further comprising determining the perfusion imaging plane.

6. The method of claim 1, where the imaging target area comprises cerebrovascular anatomy.

7. The method of claim 6, where the cerebrovascular anatomy comprises a portion of the cerebellum.

8. The method of claim 6, where the cerebrovascular anatomy comprises a portion of the hippocampus.

9. The method of claim 1, further comprising:
positioning label and control planes for magnetically tagging inflowing arterial blood of the proximal arteries of the image target area; and
adjusting for direct radio frequency spillover in the label and control planes;
where the imaging target area comprises body regions or organs in which the direction of arterial flow in labeled input arteries are not parallel to the direction of inflow to the body regions or organs from the immediately proximal arteries.

10. A system comprising:
an imaging unit; and
a controller unit, where the controller unit is configured to be operative to:
position a perfusion imaging plane that corresponds to an image target area of an imaging object, where the perfusion imaging plane is orthogonal to a direction of inflow from immediately proximal arteries of the image target area; and
cause an MRI image to be generated that corresponds to a representation of the image target area at the perfusion imaging plane.

11. The system of claim 10, where the controller unit is configured to be further operative to receive data corresponding to the direction of inflow.

12. The system of claim 10, where the controller unit is configured to be further operative to determine the direction of inflow.

13. The system of claim 10, where the controller unit is configured to be further operative to receive data corresponding to the perfusion imaging plane.

14. The system of claim 10, where the controller unit is configured to be further operative to determine the perfusion imaging plane.

15. The system of claim 14, where the imaging target area comprises cerebrovascular anatomy.

16. The system of claim 15, where the cerebrovascular anatomy comprises a portion of the cerebellum.

17. The system of claim 15, where the cerebrovascular anatomy comprises a portion of the hippocampus.

18. A computer readable medium having computer usable program code executable to perform operations comprising:
determining a perfusion imaging plane that corresponds to an image target area of an imaging object, where the perfusion imaging plane is orthogonal to a direction of inflow from immediately proximal arteries of the image target area; and
sending an output that is configured to cause an MRI image to be generated that corresponds to a representation of the image target area at the perfusion imaging plane.

19. The computer readable medium of claim 18, the operations further comprising receiving data corresponding to the direction of inflow.

20. The computer readable medium of claim 18, the operations further comprising determining the direction of inflow.

21. The computer readable medium of claim 18, the operations further comprising receiving data corresponding to the perfusion imaging plane.

22. The computer readable medium of claim 18, the operations further comprising determining the perfusion imaging plane.

23. The computer readable medium of claim 18, where the imaging target area comprises cerebrovascular anatomy.

24. The computer readable medium of claim 23, where the cerebrovascular anatomy comprises a portion of the cerebellum.

25. The computer readable medium of claim 23, where the cerebrovascular anatomy comprises a portion of the hippocampus.

26. The computer readable medium of claim 23, where the cerebrovascular anatomy comprises body regions or organs in which the direction of arterial flow in labeled input arteries are not parallel to the direction of inflow to the body regions or organs from the immediately proximal arteries.
